(19) United States
(12) Patent Application Publication
(10) Pub. No.: US 2022/0204918 A1
(43) Pub. Date: Jun. 30, 2022

(54) CELL CULTURE MEDIA COMPRISING KETO ACIDS

(71) Applicant: MERCK PATENT GMBH, DARMSTADT (DE)

(72) Inventors: Aline ZIMMER, Gross-Gerau (DE); Ronja SEIBEL, Darmstadt (DE); Corinna SCHMIDT, Griesheim (DE); Gregor Franz Werner WILLE, Buchs (DE); Markus Klaus Robert FISCHER, Diepoldsau (CH)

(73) Assignee: MERCK PATENT GMBH, DARMSTADT (DE)

(21) Appl. No.: 17/602,458

(22) PCT Filed: Apr. 8, 2020
(86) PCT No.: PCT/EP2020/059950

§ 371 Date: Oct. 8, 2021

(30) Foreign Application Priority Data
Apr. 11, 2019 (EP) 19168683.1

(51) Int. Cl. C12N 5/00

(52) U.S. Cl. CPC C12N 5/0018; C12N 2500/30

(57) ABSTRACT

The present invention relates to cell culture media comprising alpha keto acids. The poor solubility of some amino acids like isoleucine, leucine and valine can be overcome by substituting them with the respective alpha keto acid.

The present invention relates to cell culture media comprising alpha keto acids. The poor solubility of some amino acids like isoleucine, leucine and valine can be overcome by substituting them with the respective alpha keto acid.

Cell culture media support and maintain the growth of cells in an artificial environment.

Depending on the type of organism whose growth shall be supported, the cell culture media comprise a complex mixture of components, sometimes more than one hundred different components.

The cell culture media required for the propagation of mammalian, insect or plant cells are typically much more complex than the media to support the growth of bacteria and yeasts.

The first cell culture media that were developed consisted of undefined components, such as plasma, serum, embryo extracts, or other non-defined biological extracts or peptones. A major advance was thus made with the development of chemically defined media. Chemically defined media often comprise but are not exclusively limited to amino acids, vitamins, metal salts, antioxidants, chelators, growth factors, buffers, hormones, and many more substances known to those expert in the art.

Some cell culture media are offered as sterile aqueous liquids. The disadvantage of liquid cell culture media is their reduced shelf life and difficulties for shipping and storage. As a consequence, many cell culture media are presently offered as finely milled dry powder mixtures. They are manufactured for the purpose of dissolving in water and/or aqueous solutions and in the dissolved state are designed, often with other supplements, for supplying cells with a substantial nutrient base for growth and/or production of biopharmaceuticals from said cells.

Many biopharmaceutical production platforms are based on fed-batch cell culture protocols. The aim typically is to develop high-titer cell culture processes to meet increasing market demands and reduce manufacturing costs. Beside the use of high-performing recombinant cell lines, improvements in cell culture media and process parameters are required to realize the maximum production potentials.

In a fed-batch process, a basal medium supports initial growth and production, and a feed medium prevents depletion of nutrients and sustains the production phase. The media are chosen to accommodate the distinct metabolic requirements during different production phases. Process parameter settings—including feeding strategy and control parameters—define the chemical and physical environments suitable for cell growth and protein production.

Optimization of the feed medium is a major aspect in the optimization of a fed-batch process.

Mostly the feed medium is highly concentrated to avoid dilution of the recombinant protein in the bioreactor. The controlled addition of the nutrient directly affects the growth rate of the culture, the viability as well as the titer.

But also in other cell culture processes like batch process or perfusion processes there is a need for precisely composed and often highly concentrated media formulations. In particular in perfusion processes, the constant exchange of the medium in the bioreactor requires operators to prepare and handle huge volumes of liquid medium. To reduce the footprint necessary to store these volumes, concentration of the media is required.

A limiting factor for the preparation of cell culture media from dry powder is the poor solubility or stability of some components, especially of some amino acids.

Consequently it would be favourable to find a way to provide dry powder media compositions which are sufficiently soluble to generate highly concentrated liquid media compositions.

It has been found that instead of the amino acids isoleucine, leucine, valine, phenylalanine and methionine, the respective alpha keto acids can be used without any negative effect and sometimes even with positive effect on the cell growth and an improved solubility.

It has further been found that those keto acids even have a stabilizing effect on liquid cell culture media formulations.

In 1959 in a paper dealing with the metabolism of amino acids it was stated that some amino acids can be substituted by their keto acids

(Eagle H: Amino acid metabolism in mammalian cell cultures. Science 1959, 130(3373):432-437.). But it has never been noticed since, that certain keto acids could be used as an amino acid substitute in high performance cell culture and that they are suitable to overcome the solubility and stability problems of some amino acids.

The present invention is therefore directed to a dry powder or dry, granulated cell culture medium comprising at least one alpha keto acid out of the group of 4-Methyl-2-oxopentanoic acid (keto Leu), 3-methyl-2-oxopentanoic acid (keto Ile), alpha-ketoisovaleric acid (keto Val), phenylpyruvic acid (keto Phe) and alpha keto gamma methylthiobutyric acid (keto Met), and/or derivatives thereof in an amount so that the concentration of each keto acid and/or derivative thereof in the liquid medium obtained after dissolution of the dry powder or dry, granulated cell culture medium is above 10 mM, preferably between 20 and 600 mM, most preferred between 30 and 300 mM. Typically each keto acid is present in a different concentration, whereby typically 4-Methyl-2-oxopentanoic acid (keto Leu), 3-methyl-2-oxopentanoic acid (keto Ile), alpha-ketoisovaleric acid (keto Val) and phenylpyruvic acid (keto Phe) and/or derivatives thereof are present in higher concentrations above 50 mM whereby alpha keto gamma methylthiobutyric acid (keto Met) is typically present in lower concentrations, typically between 10 and 30 mM.

In a preferred embodiment, if the dry powder or dry, granulated cell culture medium is a feed medium it comprises less than 30 mol % of the corresponding amino acid compared to the keto acid and/or derivatives. That means the molar ratio of the two compounds is less than 3:10.

In another embodiment the dry powder or dry, granulated cell culture feed medium does not comprise the corresponding amino acid.

For other media like perfusion media or (fed)batch basic media it might be favourable to have both, the amino acid and the corresponding keto acid and/or derivatives thereof in the medium formulation.

In another embodiment, the dry powder or dry, granulated cell culture medium comprises two or more of the alpha keto acids and/or their derivatives.

In a preferred embodiment, the dry powder or dry, granulated cell culture medium comprises the sodium salt of one or more of the alpha keto acids listed above.

In a preferred embodiment, the dry powder or dry, granulated cell culture medium comprises one or more of the alpha keto acids selected from 4-Methyl-2-oxopentanoic acid, 3-methyl-2-oxopentanoic acid, alpha-ketoisovaleric acid and/or salts thereof, preferably the sodium salts thereof.

The present invention is further directed to a method for stabilizing a liquid cell culture medium comprising including in the medium at least 20 mM, preferably between 30 and 600 mM of one or more of the alpha keto acids selected from 4-Methyl-2-oxopentanoic acid, 3-methyl-2-oxopentanoic acid, alpha-ketoisovaleric acid and/or derivatives thereof, preferably 4-Methyl-2-oxopentanoic acid and/or 3-methyl-2-oxopentanoic acid and/or alpha-ketoisovaleric acid and/or derivatives thereof and whereby the resulting medium shows less change in color and/or less precipitation upon storage over 90 days at 4° C. or room temperature compared to a medium of the otherwise same composition which lacks the keto acids and/or a derivatives thereof or in which the keto acids and/or derivatives thereof have been substituted by the corresponding amino acids and/or derivatives thereof.

The present invention is further directed to a method for improving the solubility of a dry powder or dry, granulated cell culture medium of a defined composition by fully or partially substituting one or more of the amino acids isoleucine, leucine, valine, phenylalanine and methionine by the corresponding keto acid selected from the group of 4-Methyl-2-oxopentanoic acid, 3-methyl-2-oxopentanoic acid, alpha-ketoisovaleric acid, phenylpyruvic acid and alpha keto gamma methylthiobutyric acid and/or derivatives thereof.

In a preferred embodiment at least 50%, more preferred 70%, most preferred at least 90% (molar ratio) of the respective amino acid is substituted by the corresponding alpha keto acid and/or derivatives thereof.

Substituting in this case means that instead of a given amount of an amino acid at least 80 mol %, typically around 100 mol %, of the corresponding keto acid and/or derivatives thereof are added to the medium. Preferably, between 100 and 150 mol % of the corresponding keto acid and/or derivatives thereof are added to the medium.

In a preferred embodiment the method involves providing the dry powder or dry, granulated cell culture medium in which the amino acids have been substituted as explained above and dissolving said medium whereby dissolution occurs faster and/or in less liquid compared to a medium of otherwise identical composition in which the amino acids have not been substituted.

In another preferred embodiment the dry powder or dry, granulated medium is dissolved to give a liquid medium having a pH of 8.5 or less.

In a preferred embodiment it is dissolved to give a liquid medium with a pH between 6.5 and 8.5, most preferred between 6.7 and 7.8.

In one embodiment, the dry powder or dry granulated cell culture medium whose solubility is improved comprises at least one or more saccharide components, one or more amino acids, one or more vitamins or vitamin precursors, one or more salts, one or more buffer components, one or more co-factors and one or more nucleic acid components.

In another embodiment the dry powder or dry granulated cell culture medium whose solubility is improved is dissolved to result in a liquid medium which comprises between 50 and 400 g/I, preferably between 100 and 300 g/l of solid ingredients that are dissolved in the solvent and/or the concentration of each keto acid and/or its salts is above 10 mM, preferably between 30 and 600 mM.

The present invention is further directed to a method for producing a dry powder cell culture medium according to the present invention by
a) mixing at least one alpha keto acid out of the group of 4-Methyl-2-oxopentanoic acid, 3-methyl-2-oxopentanoic acid, alpha-ketoisovaleric acid, phenylpyruvic acid and alpha keto gamma methylthiobutyric acid and/or derivatives thereof with the other components of the cell culture medium
b) subjecting the mixture of step a) to milling

In a preferred embodiment step b) is performed in a pin mill, fitz mill or a jet mill.

In another preferred embodiment, the mixture from step a) is cooled to a temperature below 0° C. prior to milling.

The present invention is further directed to a process for culturing cells by

a) providing a bioreactor
b) mixing the cells to be cultured with a liquid cell culture medium in which one or more of the amino acids isoleucine, leucine, valine, phenylalanine and methionine are partially or fully substituted by the corresponding keto acid selected from the group of 4-Methyl-2-oxopentanoic acid, 3-methyl-2-oxopentanoic acid, alpha-ketoisovaleric acid, phenylpyruvic acid and alpha keto gamma methylthiobutyric acid, and/or derivatives thereof.
c) incubating the mixture of step b).

In a preferred embodiment the liquid cell culture medium comprises each keto acid and/or its derivative that is present in a concentration of more than 10 mM.

The present invention is also directed to a fed batch process for culturing cells in a bioreactor by
Filling into a bioreactor cells and an aqueous cell culture medium
Incubating the cells in the bioreactor
Continuously over the whole time of the incubation of the cells in the bioreactor or once or several times within said incubation time adding a cell culture medium, which is in this case a feed medium, to the bioreactor
   whereby the feed medium has a pH of less than pH 8.5 and comprises at least one alpha keto acid out of the group of 4-Methyl-2-oxopentanoic acid, 3-methyl-2-oxopentanoic acid, alpha-ketoisovaleric acid, phenylpyruvic acid and alpha keto gamma methylthiobutyric acid and/or derivatives thereof.

Preferably the feed medium comprises at least 4-Methyl-2-oxopentanoic acid, 3-methyl-2-oxopentanoic acid, alpha-ketoisovaleric acid and/or salts thereof in a concentration of each between 20 and 600 mmol/l, preferably between 20 and 400 mmol/l.

The present invention is further directed to a perfusion process with a liquid cell culture medium in which one or more of the amino acids isoleucine, leucine, valine, phenylalanine and methionine are partially or fully substituted by the corresponding keto acid selected from the group of 4-Methyl-2-oxopentanoic acid, 3-methyl-2-oxopentanoic acid, alpha-ketoisovaleric acid, phenylpyruvic acid and alpha keto gamma methylthiobutyric acid, and/or derivatives thereof.

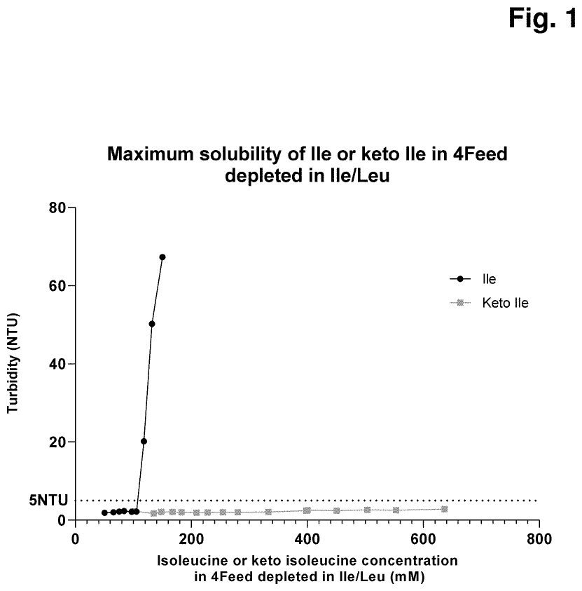

FIG. 1 shows the determination of the maximum solubility of Ile or keto Ile in a Cellvento® 4Feed formulation depleted in Ile and Leu (125 g/L, pH 7.0+/−0.2). A solution having a turbidity below 5 NTU is considered soluble.

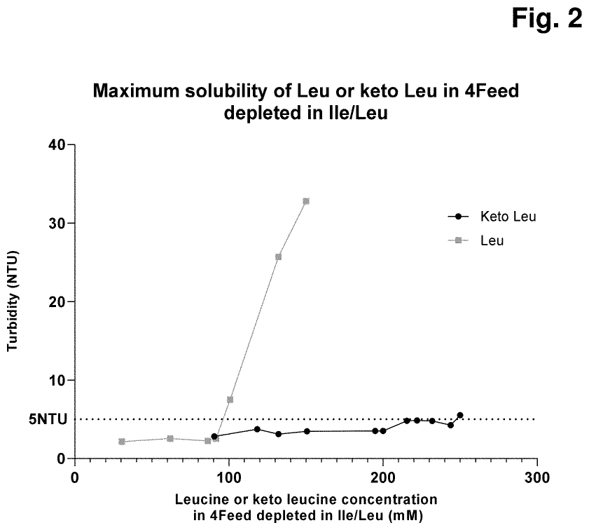

FIG. 2 shows the determination of the maximum solubility of Leu or keto Leu in a Cellvento® 4Feed formulation depleted in Ile and Leu (125 g/L, pH 7.0+/−0.2). A solution having a turbidity below 5 NTU is considered soluble. Further information about FIGS. 1 and 2 can be found in Example 2.

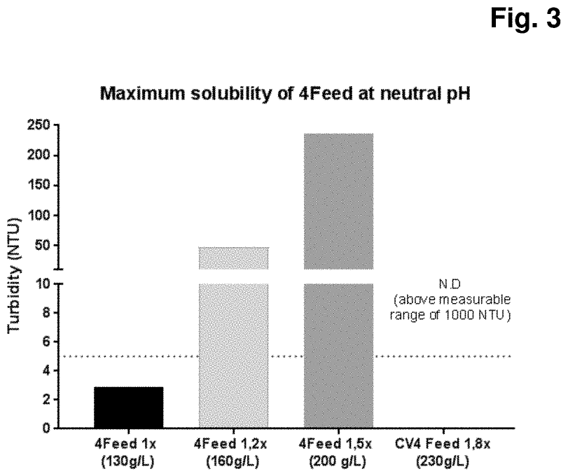

FIG. 3 shows the solubility limit of Cellvento® 4Feed at pH 7.0. Turbidity was measured using a turbidometer. Further details can be found in Example 3.

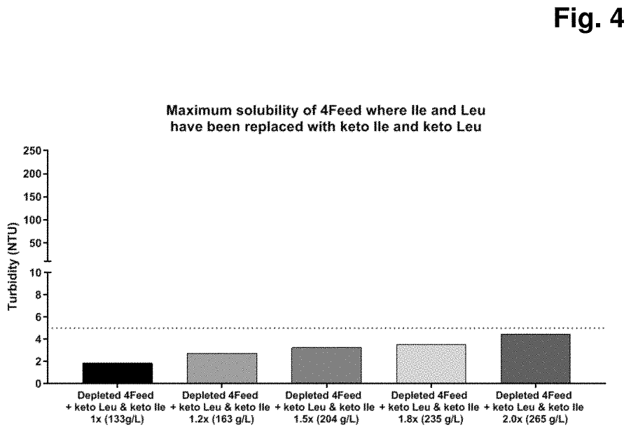

FIG. 4 shows the solubility limit of a modified 4Feed formulation where Ile and Leu have been replaced by keto Ile and keto Leu at pH 7.0. Turbidity was measured using a turbidometer. Further details can be found in Example 3.

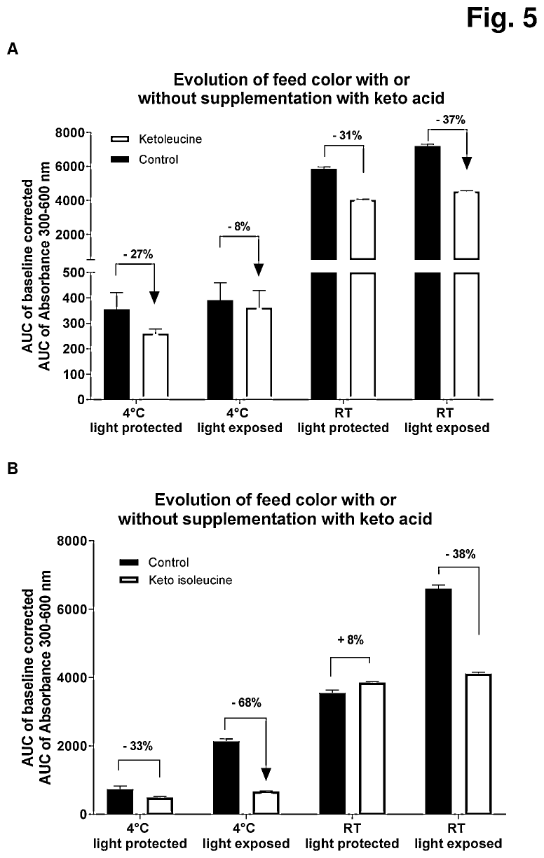

FIG. 5A shows the AUC over time (D0 to D90) of the baseline corrected area under the curve of the absorbance between 300 and 600 nm for the control feed containing Leu and the test feed depleted in Leu and replaced with equimolar concentration of keto Leu.

FIG. 5B shows the AUC over time (D0 to D90) of the baseline corrected area under the curve of the absorbance between 300 and 600 nm for the control feed containing isoleucine and the test feed depleted in ile and replaced with equimolar concentrations of keto Ile. Details can be found in Example 4.

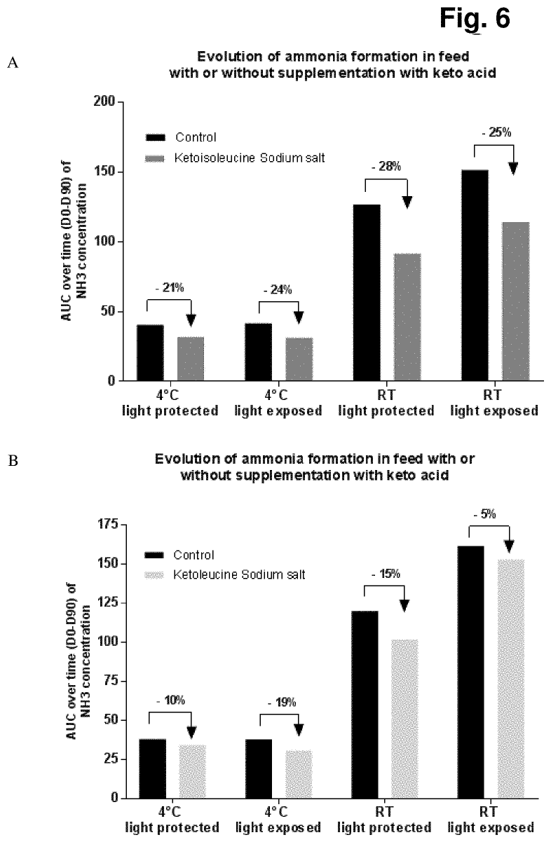

FIG. 6A shows the area under the curve (D0 to D90) of the NH₃ concentration measured in a feed containing keto Leu compared to the control. The feed was stored at 4° C. and RT and either protected or exposed to light for 3 months.

FIG. 6B shows the area under the curve (D0 to D90) of the NH₃ concentration measured in a feed containing keto Ile compared to the control. The feed was stored at 4° C. and RT and either protected or exposed to light for 3 months. Details can be found in Example 4.

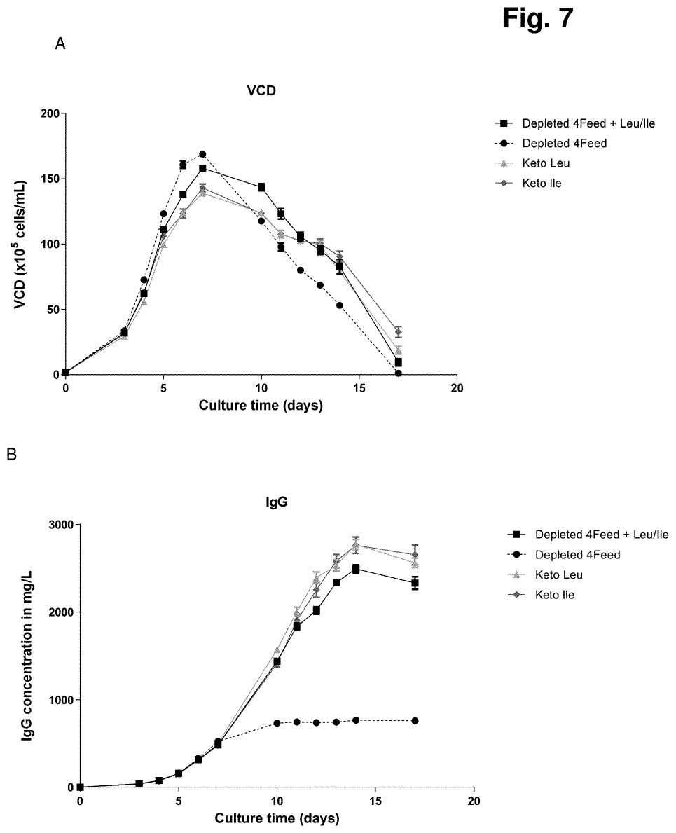

FIG. 7A: VCD over a 17-days fed-batch process with either keto Leu or keto Ile replacing Leu and Ile respectively in the feed. Depleted 4Feed is the negative control and does not contain any Leu or Ile.

FIG. 7B: IgG produced over a 17-days fed-batch process with either keto Leu or keto Ile replacing Leu and Ile respectively in the feed. Details can be found in Example 5.

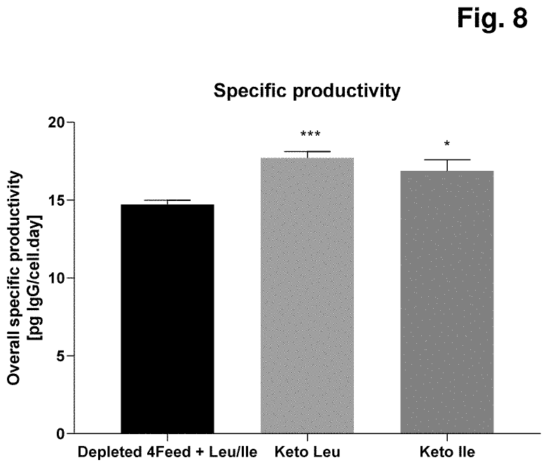

FIG. 8: Averaged specific productivity for a 17-days fed-batch process with either keto Leu or keto Ile replacing Leu and Ile respectively in the feed.

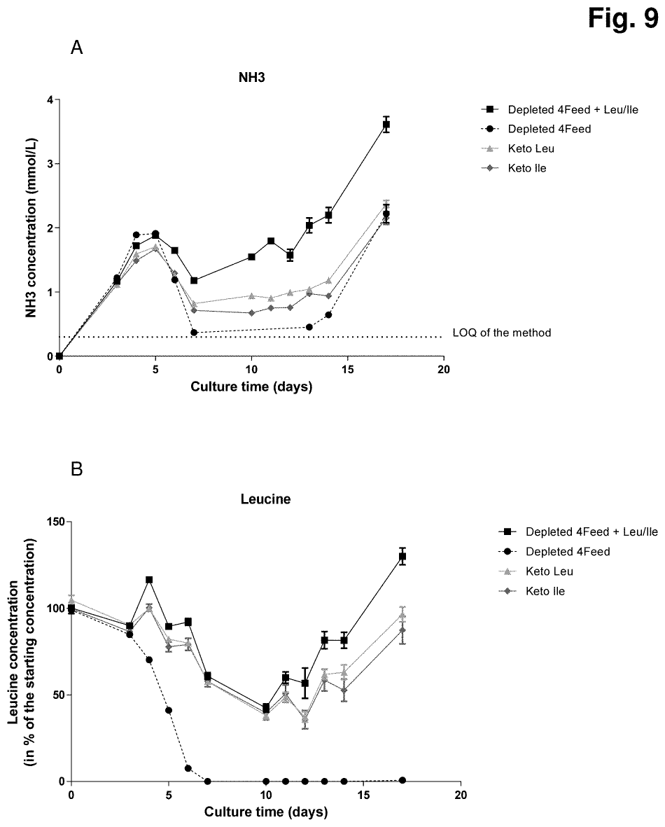

FIG. 9A: NH₃ production during a 17-days fed-batch process with either keto Leu or keto Ile replacing Leu and Ile respectively in the feed.

FIG. 9B: Leu quantification in the spent medium during a 17-days fed-batch process with either keto Leu or keto Ile replacing Leu and Ile respectively in the feed.

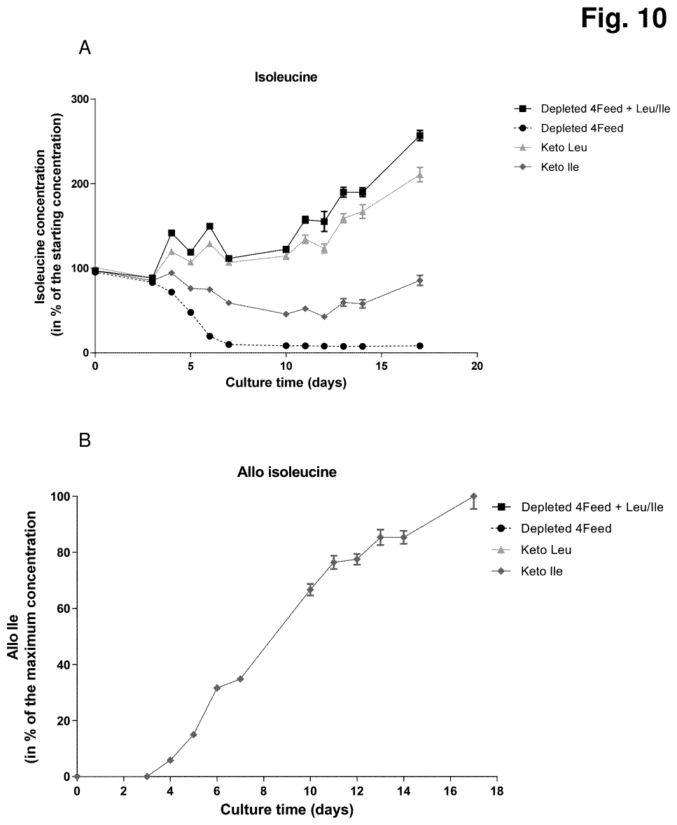

FIG. 10A: Ile quantification in the spent medium during a 17-days fed-batch process with either keto Leu or keto Ile replacing Leu and Ile respectively in the feed.

FIG. 10B: allo-Ile quantification in the spent medium during a 17-days fed-batch process with keto Ile replacing Ile in the feed. Further details can be found in Example 5.

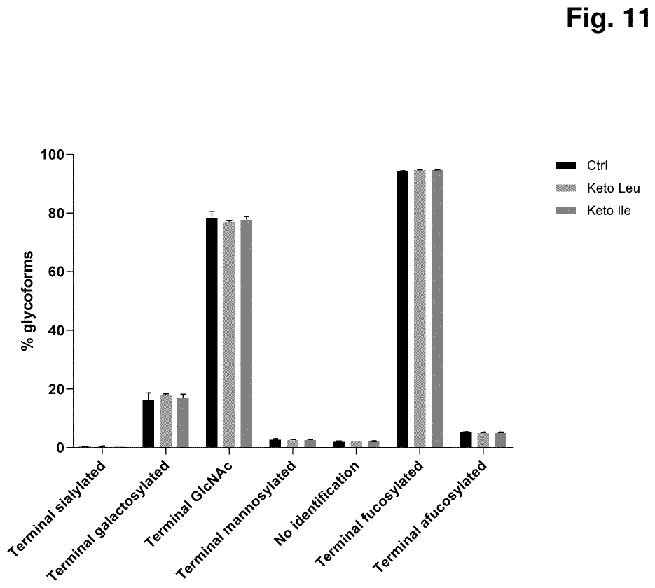

FIG. 11: Glycosylation of an IgG1 produced in the control process or in a process where a feed depleted in Ile/Leu and supplemented with either keto Leu or keto Ile was used. Glycoform distribution was determined using APTS labeling and CGE-LIF detection.

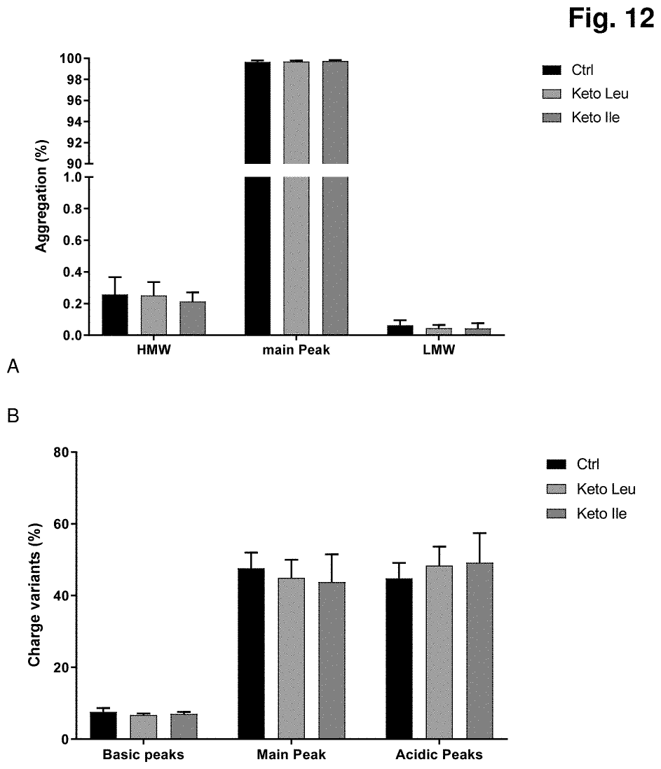

FIG. 12A: Aggregation and fragmentation of an IgG1 produced in the control process or in a process where a feed depleted in Ile/Leu and supplemented with either keto Leu or keto Ile was used. High molecular weight (HMW) and low molecular weight species (LMW) were determined using size exclusing chromatography.

FIG. 12B: Charge variants of an IgG1 produced in the control process or in a process where a feed depleted in Ile/Leu and supplemented with either keto Leu or keto Ile was used. Charge variant distribution was determined using cIEF on a Capillary Electrophoresis CESI 8000.

Further details can be found in Example 5.

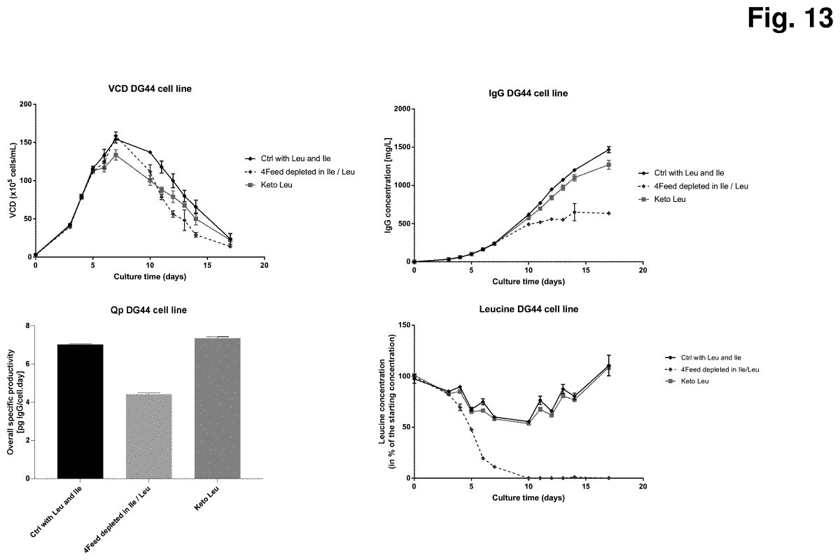

FIG. 13: Performance of the keto Leu containing process compared to the control for a CHODG44 cell line expressing an IgG1.

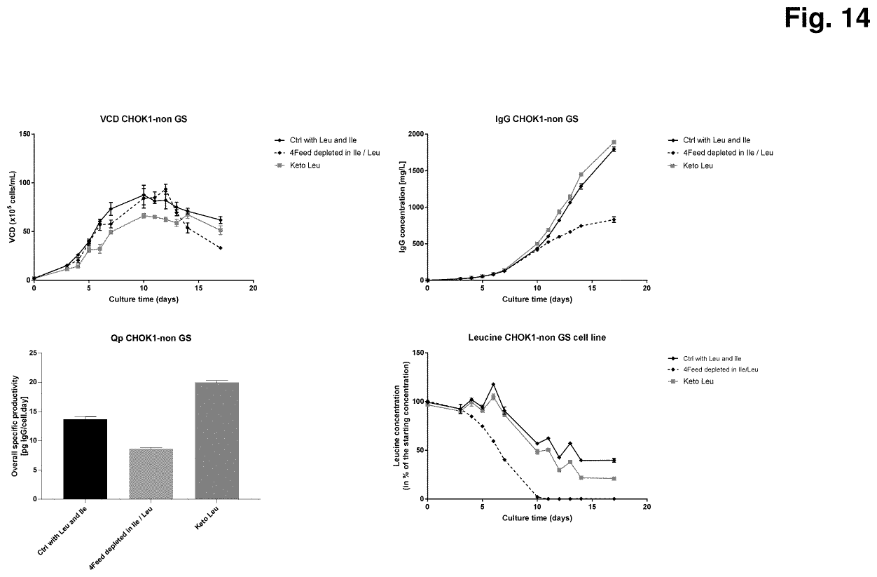

FIG. 14: Performance of keto Leu containing process compared to the control for a CHOK1 non GS cell line expressing an IgG1. Further details can be found in Example 6.

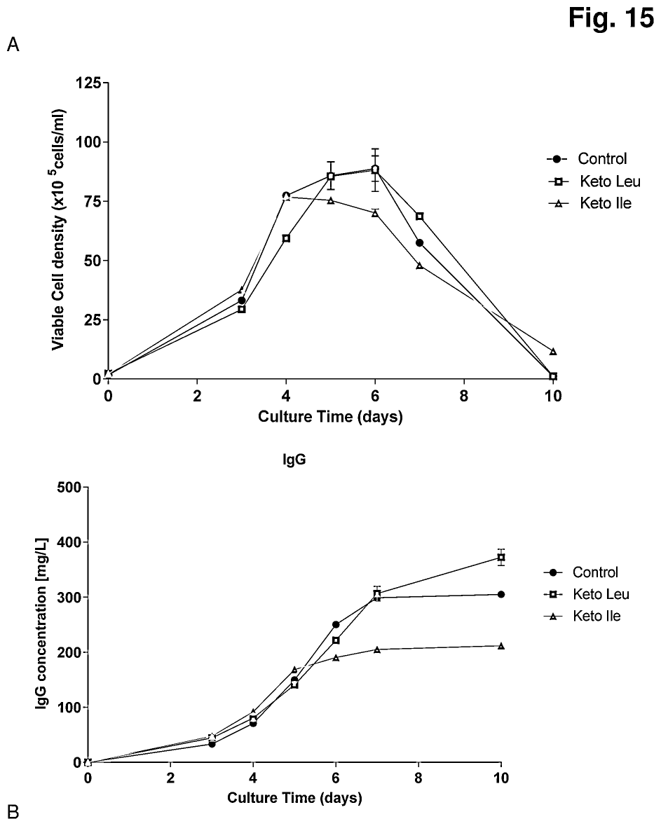

FIG. 15A: Batch experiment with a CHOK1GS cell line cultivated in a medium containing Leu and Ile (Ctrl) or a medium where Ile or Leu have been replaced by their equimolar concentration of keto Ile or keto Leu.

Seeding density was 0.2 million cells/mL, VCD was measured using Vi-CELL XR.

FIG. 15B: IgG concentration measured during the batch experiment. IgG was measured using a turbidometric assay on the Cedex Bio HT (Roche). Further details can be found in Example 7.

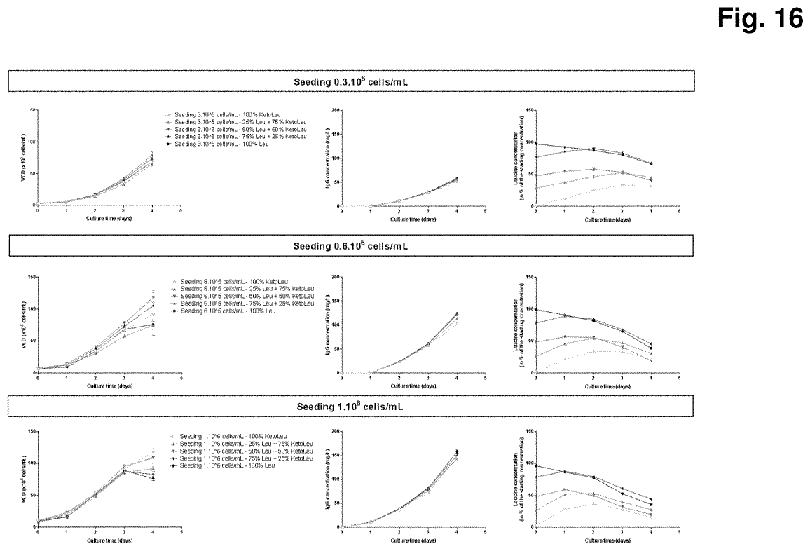

FIG. 16: Batch experiments with higher seeding densities and different Leu/keto Leu ratios. VCD and titer were measured as well as the released leucine in the spent medium. Further details can be found in Example 7.

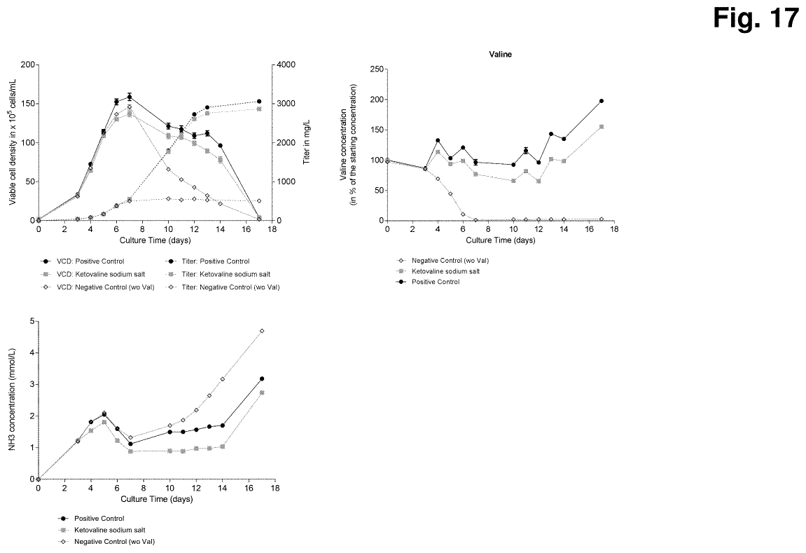

FIG. 17: Replacement of Val with keto Val in the feed. VCD and titer were measured as well as the released Val and NH₃ concentrations in the spent medium. Further details can be found in Example 8.

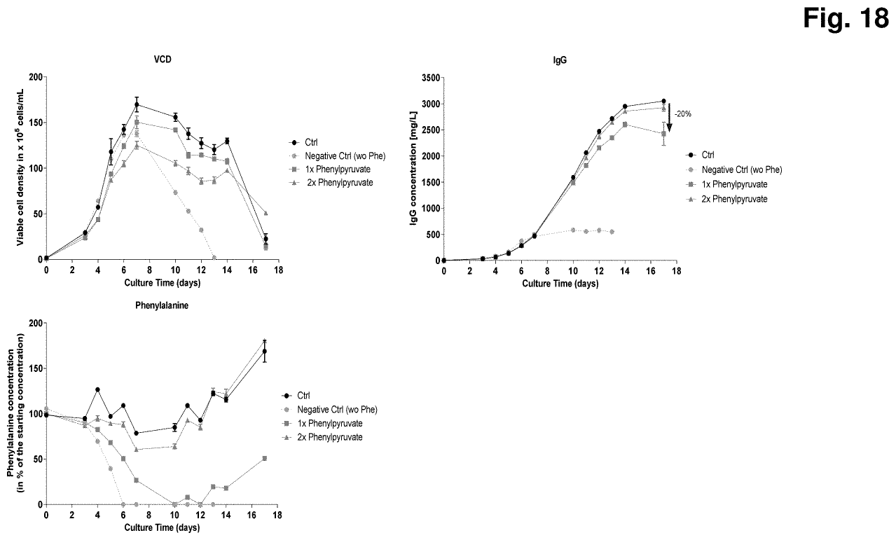

FIG. 18: Replacement of Phe with phenylpyruvate in the feed with either the same molar concentration (1×) or the doubled concentration compared to Phe (2×). VCD and titer were measured as well as the released Phe concentration in the spent medium. Further details can be found in Example 8.

A cell culture medium according to the present invention is any mixture of components which maintains and/or supports the in vitro growth of cells. It might be a complex medium or a chemically defined medium. The cell culture medium can comprise all components necessary to maintain and/or support the in vitro growth of cells or only some components so that further components are added separately. Examples of cell culture media according to the present invention are full media which comprise all components necessary to maintain and/or support the in vitro growth of cells as well as media supplements or feeds. In a preferred embodiment the cell culture medium is a full medium, a perfusion medium or a feed medium. A full medium also called basal medium typically has a pH between 6.7 and 7.8. A feed medium preferably has a pH below 8.5.

Typically, the cell culture media according to the invention are used to maintain and/or support the growth of cells in a bioreactor.

A feed or feed medium is a cell culture medium which is not the basal medium that supports initial growth and production in a cell culture but the medium which is added at a later stage to prevent depletion of nutrients and sustains the production phase. A feed medium can have higher concentrations of some components compared to a basal culture medium. For example, some components, such as, for example, nutrients including amino acids or carbohydrates, may be present in the feed medium at about 5×, 6×, 7×, 8×, 9×, 10×, 12×, 14×, 16×, 20×, 30×, 50×, 100×, 200×, 400×, 600×, 800×, or even about 1000× of the concentrations in a basal medium.

A mammalian cell culture medium is a mixture of components which maintain and/or support the in vitro growth of mammalian cells. Examples of mammalian cells are human or animal cells, preferably CHO cells, COS cells, I VERO cells, BHK cells, AK-1 cells, SP2/0 cells, L5.1 cells, hybridoma cells or human cells.

Chemically defined cell culture media are cell culture media that do not comprise any chemically undefined substances. This means that the chemical composition of all the chemicals used in the media is known. The chemically defined media do not comprise any yeast, animal or plant tissues; they do not comprise feeder cells, serum, hydrolysates, extracts or digests or other poorly defined components. Chemically undefined or poorly defined chemical components are those whose chemical composition and structure is not known, are present in varying composition or could only be defined with enormous experimental effort—comparable to the evaluation of the chemical composition and structure of a protein like insulin, albumin or casein.

A powdered cell culture medium or a dry powder medium is a cell culture medium typically resulting from a milling process or a lyophilisation process. That means the powdered cell culture medium is a granular, particulate medium—not a liquid medium. The term “dry powder” may be used interchangeably with the term “powder;” however, “dry powder” as used herein simply refers to the gross appearance of the granulated material and is not intended to mean that the material is completely free of complexed or agglomerated solvent unless otherwise indicated.

A dry, granulated medium is a dry medium resulting from a wet or dry granulation process. Preferably, it is a medium resulting from roller compaction of a dry powder medium. The term dry as used herein simply refers to the gross appearance of the granulated material and is not intended to mean that the material is completely free of complexed or agglomerated solvent unless otherwise indicated.

Cells to be cultured with the media according to the present invention may be prokaryotic cells like bacterial cells or eukaryotic cells like plant or animal cells. The cells can be normal cells, immortalized cells, diseased cells, transformed cells, mutant cells, somatic cells, germ cells, stem cells, precursor cells or embryonic cells, any of which may be established or transformed cell lines or obtained from natural sources.

The size of a particle means the mean diameter of the particle. The particle diameter is determined by laser light scattering (Mastersizer 3000, Malvern).

A change in color of a liquid cell culture medium is preferably determined visually or spectroscopically.

Precipitation can be determined visually or by turbidometric methods.

An inert atmosphere is generated by filling the respective container or apparatus with an inert gas. Suitable inert gases are noble gases like argon or preferably nitrogen. These inert gases are non-reactive and prevent undesirable chemical reactions from taking place. In the process according to the present invention, generating an inert atmosphere means that the concentration of oxygen is reduced below 10% (v/v) absolute, e.g. by introducing liquid nitrogen or nitrogen gas.

Different types of mills are known to a person skilled in the art. A pin mill, also called centrifugal impact mill, pulverizes solids whereby protruding pins on high-speed rotating disks provide the breaking energy. Pin mills are for example sold by Munson Machinery (USA), Premium Pulman (India) or Sturtevant (USA).

A jet mill uses compressed gas to accelerate the particles, causing them to impact against each other in the process chamber. Jet mills are e.g. sold by Sturtevant (USA) or PMT (Austria).

A fitz mill commercialized by Fitzpatrick (USA), uses a rotor with blades for milling.

A process that is run continuously is a process that is not run batchwise. If a milling process is run continuously it means that the media ingredients are permanently and steadily fed into the mill over a certain time.

The cell culture media, especially the full media, according to the present invention typically comprise at least one or more saccharide components, one or more amino acids, one or more vitamins or vitamin precursors, one or more salts, one or more buffer components, one or more co-factors and one or more nucleic acid components.

The media may also comprise sodium pyruvate, insulin, vegetable proteins, fatty acids and/or fatty acid derivatives and/or pluronic acid and/or surface active components like chemically prepared non-ionic surfactants. One example of a suitable non-ionic surfactant are difunctional block copolymer surfactants terminating in primary hydroxyl groups also called poloxamers, e.g. available under the trade name Pluronic® from BASF, Germany.

Saccharide components are all mono- or di-saccharides, like glucose, galactose, ribose or fructose (examples of monosaccharides) or sucrose, lactose or maltose (examples of disaccharides).

Examples of amino acids according to the invention are tyrosine, the proteinogenic amino acids, especially the essential amino acids, leucine, isoleucine, lysine, methionine, phenylalanine, arginine, threonine, tryptophane and valine, as well as the non-proteinogenic amino acids like D-amino acids, whereby the L-amino acids are preferred. The term of the amino acids further includes the salts of the amino acids, like the sodium salts, or the respective hydrates or hydrochlorides.

For example, tyrosine means L- or D-tyrosine, preferably L-tyrosine, as well as its salts or hydrates or hydrochlorides.

Examples of vitamins are Vitamin A (Retinol, retinal, various retinoids, and four carotenoids), Vitamin B₁ (Thiamine), Vitamin B₂ (Riboflavin), Vitamin B₃ (Niacin, niacinamide), Vitamin B₅ (Pantothenic acid), Vitamin B₆ (Pyridoxine, pyridoxamine, pyridoxal), Vitamin B₇ (Biotin), Vitamin B₉ (Folic acid, folinic acid), Vitamin B₁₂ (Cyanocobalamin, hydroxycobalamin, methylcobalamin), Vitamin C (Ascorbic acid), Vitamin D (Ergocalciferol, cholecalciferol), Vitamin E (Tocopherols, tocotrienols) and Vitamin K (phylloquinone, menaquinones). Vitamin precursors are also included.

Examples of salts are components comprising inorganic ions such as bicarbonate, calcium, chloride, magnesium, phosphate, potassium and sodium or trace elements such as Co, Cu, F, Fe, Mn, Mo, Ni, Se, Si, Ni, Bi, V and Zn. Examples are Copper(II) sulphate pentahydrate (CuSO₄.5H₂O), Sodium Chloride (NaCl), Calcium chloride (CaCl₂.2H₂O), Potassium chloride (KCl), Iron(II)sulphate, ferric ammonium citrate (FAC), sodium phosphate monobasic anhydrous (NaH₂PO₄), Magnesium sulphate anhydrous (MgSO₄), sodium phosphate dibasic anhydrous (Na₂HPO₄), Magnesium chloride hexahydrate (MgCl₂.6H₂O), zinc sulphate heptahydrate.

Examples of buffers are CO₂/HCO₃ (carbonate), phosphate, HEPES, PIPES, ACES, BES, TES, MOPS and TRIS.

Examples of cofactors are thiamine derivatives, biotin, vitamin C, NAD/NADP, cobalamin, flavin mononucleotide and derivatives, glutathione, heme nucleotide phophates and derivatives.

Nucleic acid components, according to the present invention, are the nucleobases, like cytosine, guanine, adenine, thymine or uracil, the nucleosides like cytidine, uridine, adenosine, guanosine and thymidine, and the nucleotides like adenosine monophosphate or adenosine diphosphate or adenosine triphosphate.

Feed media may have a different composition compared to full media. They typically comprise amino acids, trace elements and vitamins. They might also comprise saccharide components but sometimes for production reasons the saccharide components are added in a separate feed.

A suitable feed medium might for example comprise one or more of the following compounds:
L-ASPARAGINE MONOHYDRATE
L-ISOLEUCINE
L-PHENYLALANINE
SODIUM L-GLUTAMATE MONOHYDRATE
L-LEUCINE
L-THREONINE
L-LYSINE MONOHYDROCHLORIDE
L-PROLINE
L-SERINE
L-ARGININE MONOHYDROCHLORIDE
L-HISTIDINE MONOHYDROCHLORIDE MONOHYDRATE
L-METHIONINE
L-VALINE
MONO-SODIUM-L-ASPARTATE-MONOHYDRATE
L-TRYPTOPHAN
CHOLINE CHLORIDE
MYO-INOSITOL
NICOTINAMIDE
CALCIUM-D(+) PANTOTHENATE
PYRIDOXINE HYDROCHLORIDE
THIAMINE CHLORIDE HYDROCHLORIDE
VITAMIN B12 (CYANOCOBALAMINE) MICRONIZED
BIOTIN
FOLIC ACID
RIBOFLAVIN
MAGNESIUM SULFATE ANHYDROUS
COPPER(II) SULFATE PENTAHYDRATE
ZINC SULFATE HEPTAHYDRATE
1,4-DIAMINOBUTANE DIHYDRCHLORIDE
AMMONIUM HEPTAMOLYBDATE TETRAHYDRATE
CADMIUM SULFATE HYDRATE
MANGANESE(II) CHLORIDE TETRAHYDRATE
NICKEL(II) CHLORIDE HEXAHYDRATE
SODIUM META SILICATE
SODIUM METAVANADATE
TIN(II) CHLORIDE DIHYDRATE
SODIUM SELENITE (ABOUT 45% SE)
SODIUM DIHYDROGEN PHOSPHATE MONOHYDRATE
AMMONIUM IRON(III) CITRATE (ABOUT 18% FE)

Freezing according to the present invention means cooling to a temperature below 0° C.

In a perfusion process, the cell culture medium is continuously added and removed from the bioreactors through pumps while the cells are retained in the bioreactor through a cell retention device. Advantages of perfusion is the possibility to reach very high cell densities (due to the constant medium exchange) and the possibility to produce very fragile recombinant proteins since the product can be removed from the bioreactor every day thus reducing the exposure time of the recombinant protein to high temperatures, oxidizing redox potentials or released cellular proteases.

A process for perfusion cell culture typically comprises culturing cells in a bioreactor system comprising a bioreactor with a media inlet and a harvest outlet whereby

i. continuously or one or several times, preferably continuously, during the cell culture process new cell culture medium is inserted into the bioreactor via the media inlet

ii. continuously or one or several times during the cell culture process, preferably continuously, harvest is removed from the bioreactor via the harvest outlet. The harvest typically comprises the target product produced by the cells, cells and liquid cell culture medium.

Amino acids are essential components of cell culture media since they are key to support cellular growth. In addition, amino acids are key building blocks for recombinant proteins produced using mammalian cell culture technologies. The solubility of amino acids is a limiting factor hindering the concentration of cell culture media and feed formulations. Such a concentration would be essential to develop next generation manufacturing platforms. In particular, highly concentrated formulations are required for biomanufacturing processes using inline dilution, to reduce the volume of cell culture medium which has to be stored in tanks (=reduce manufacturing footprint) or in general to reduce the volume of feed added throughout a fed-batch process and thus potentially increase the volumetric titer.

It has been found that several amino acids can be replaced in cell culture media by their keto acids or salts thereof. In addition to their use as amino acid sources, especially the sodium salts of these keto acids present a higher solubility compared to their corresponding amino acids and can therefore be used in highly concentrated formulations. Next to the solubility advantage, it has been found that the use of keto acids also allows the reduction of ammonia in cell culture, which is a known toxic and inhibitory metabolite. Furthermore, the use of certain keto acids proved to yield more stable formulations when stored at RT with a reduction in color change, a lack or a delay in precipitation and with less or delayed formation of by-products.

Keto acids of amino acids and salts thereof can thus be used in cell culture media formulations for the following applications
Application 1: to increase overall media/feed solubility
Application 2: to replace the corresponding amino acid and reduce ammonium ion/ammonia formulation in cell culture
Application 3: to increase media stability, reduce change in color and precipitation due to the storage of the formulation at 4° C. or room temperature and reduce formation of ammonia during feed storage.

Table 1 shows the amino acids leucine, isoleucine, valine, phenylalanine and methionine as well as their corresponding keto acids or the sodium salts of the corresponding keto acids. As can be seen from Table 1, the solubility of the corresponding keto acids is higher than the solubility of the amino acids.

TABLE 1
Solubility of amino acids and their respective keto acids or salts
thereof in water at 25° C. Solubility experiments are performed using a
saturated solution and residual mass determination after infrared drying.

| | | | Corresponding | | | Solubility |
|---|---|---|---|---|---|---|
| Amino acid | Chemical formula | Solubility | keto acid | Availability | Chemical formula | 25° C. in g/kg |
| Leucine | 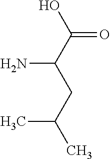 | 22.1 | 4-Methyl-2- oxopentanoic acid sodium salt | CAS 4502-00-5 Sigma W387101 (97%)/ K0629 (98%) | 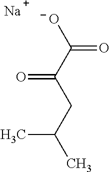 | 313.7 |
| Isoleucine | 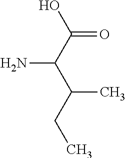 | 32.4 | 3-Methyl-2- oxopentanoic acid sodium salt | CAS 3715-31-9 Sigma 198978 (98%) | 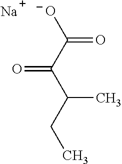 | 480 |
| Valine | 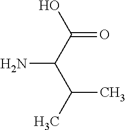 | 58.5 | α-Ketoisovaleric acid sodium salt | CAS 223-062-2 Sigma 198994 (95%) | 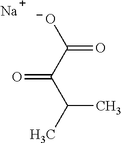 | 362 |
| Phenylalanine | 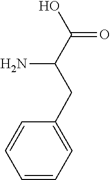 [SMILES: NC(Cc1ccccc1)C(=O)O] | 26.8 | Phenylpyruvic acid sodium salt | CAS 114-76-1 Sigma P8001 (95%) | 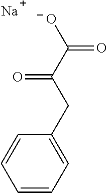 [SMILES: [Na+] ; O=C([O-])C(=O)Cc1ccccc1] | 72 |
| Methionine | 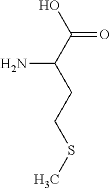 | 52.9 | Alpha keto gamma methylthiobutyric acid sodium salt | CAS 518282-97-8 Sigma K6000 (97%) | 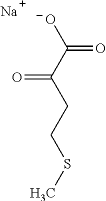 | >500 |

It has been found that by partially or fully substituting the amino acids leucine, isoleucine, valine, phenylalanine and/or methionine by the corresponding keto acid and/or derivatives thereof the solubility of a dry powder or dry, granulated cell culture medium can be improved without having a negative effect on the performance of the cell culture compared to an otherwise identical cell culture medium. In a preferred embodiment, the sodium salts of the keto acids are used as they typically show the highest solubility.

Suitable derivatives are metal salt derivatives, peptide derivatives, ester derivatives as well as other derivatives. The derivatives are keto acid derivatives and have a higher solubility in water compared to the corresponding amino acid and they are intracellularly concerted back to the corresponding amino acid or can otherwise substitute the corresponding amino acid in its role to maintain and/or support the in vitro growth of cells.

Metal salt derivatives are the most preferred derivatives. These are the metal salts of the keto acids like the sodium, potassium, calcium or magnesium salt, preferably the sodium salt.

Peptide derivatives are derivatives in which one or more, typically one, two or three amino acids are linked to the keto acid via a peptidic bond. A schematic formula of peptide derivatives in this case of keto leucine is shown in the following scheme 1:

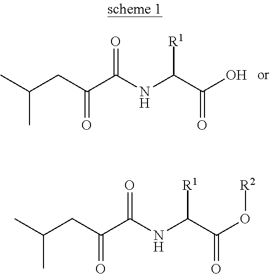

with R¹ being an amino acid side chain and R² being another amino acid linked via a peptidic bond.

Ester derivatives are derivatives in which the carboxylic acid of the keto acid forms and alkyl or aryl ester. Most preferred are C1 to C4 alkyl esters. An example of a keto-leucine ester derivative is shown in scheme 2:

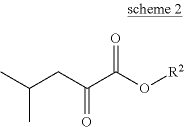

with R² being alkyl or aryl, whereby the alkyl group may be further substituted with —OH or OR² or e.g. to form an ether or an ester. Examples of suitable R² are methyl, ethyl, isopropyl, n-propyl, n-butyl, tert-butyl, benzyl as well as

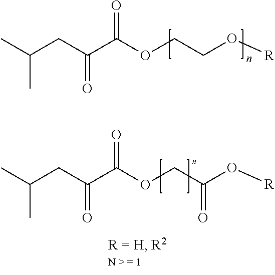

Other derivatives are shown in scheme 3:

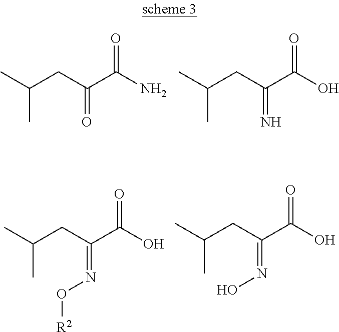

The above examples which are shown for keto leucine can of course be equally realized for the other keto acids out of the group of 4-Methyl-2-oxopentanoic acid, 3-methyl-2-oxopentanoic acid, alpha-ketoisovaleric acid, phenylpyruvic acid and alpha keto gamma methylthiobutyric acid. The present invention is therefore directed to dry powder or dry, granulated cell culture media comprising at least one alpha keto acid out of the group of 4-Methyl-2-oxopentanoic acid, 3-methyl-2-oxopentanoic acid, alpha-ketoisovaleric acid, phenylpyruvic acid and alpha keto gamma methylthiobutyric acid and/or derivatives thereof, preferably metal salt derivatives, most preferred the sodium salts. In a preferred embodiment, the dry powder or dry, granulated cell culture medium comprises the sodium salt of 4-Methyl-2-oxopentanoic acid, 3-methyl-2-oxopentanoic acid and/or alpha-ketoisovaleric acid, most preferred the sodium salts of all three keto acids.

The amount of the keto acids in the dry powder or dry, granulated cell culture medium is such that the concentration of each keto acid and/or its derivatives in the liquid medium obtained after dissolution of the dry powder or dry, granulated cell culture medium is above 10 mM, preferably between 20 and 600 mM, most preferred between 30 and 300 mM.

In one embodiment, the dry powder or dry, granulated cell culture medium comprising the keto acids as defined above does not comprise the corresponding amino acids. In another embodiment, the dry powder or dry, granulated cell culture medium comprising the keto acids as defined above comprises up to 50% (mol %) of the corresponding amino acid.

For use of the dry powder or dry, granulated media a solvent, preferably water (most particularly distilled and/or deionized water or purified water or water for injection) or an aqueous buffer is added to the media and the components are mixed until the medium is totally dissolved in the solvent.

The solvent may also comprise saline, soluble acid or base ions providing a suitable pH range (typically in the range between pH 1.0 and pH 10.0, preferably in the range between 6.5 and 8.5), stabilizers, surfactants, preservatives, and alcohols or other polar organic solvents.

It is also possible to add further substances like buffer substances for adjustment of the pH, fetal calf serum, sugars etc., to the mixture of the cell culture medium and the solvent. The resulting liquid cell culture medium is then contacted with the cells to be grown or maintained.

While dry powder or dry, granulated media compositions comprising a higher concentration of leucine, isoleucine, valine, phenylalanine and methionine would show turbidity when mixed with the solvent due to the limited solubility of the amino acids, the cell culture media according to the present invention using the same concentration of the corresponding keto acids and/or their derivatives give clear solutions. This is especially suitable for feed media.

The resulting liquid media comprising the keto acids and/or their derivatives show at least the same performance in cell culture. It has been found that the amino acids can be fully substituted by the corresponding keto acids and/or the derivatives, preferably the salts thereof. It is nevertheless also possible to only partly substitute the amino acid. In this case preferably 50% (mol %) or more of the amino acid are substituted by the corresponding keto acid and/or the derivatives thereof.

In some cases it might be advantageous to amend and especially enlarge the amount of keto acid compared to the amount of the amino acid that is substituted. While for the substitution of isoleucine, leucine and valine a 1:1 substitution is typically sufficient, it has been found that for phenylalanine and methionine it is typically preferable to add more keto acid compared to the amount of amino acid. Typically a 1:1.1 to 1:3 (on a molar basis) substitution is suitable.

By preferably fully substituting the amino acids leucine, isoleucine, valine, phenylalanine and methionine by the corresponding keto acids and/or derivatives thereof, especially by the sodium salts of the keto acids, the maximum solubility of media can be enlarged. As can be seen in Example 3 the solubility of a dry powder medium can be e.g. doubled. In addition to the improvement of the solubility of dry powder or dry, granulated media by substituting the amino acids as described above, it has further unexpectedly been found that when using media in which leucine and/or isoleucine have been replaced by the corresponding keto acid and/or salts thereof the specific productivity of the cell culture is enlarged.

It could further be shown that the three critical quality attributes of an IgG1 produced using media in which leucine and/or isoleucine have been replaced by the corresponding keto acid and/or salts thereof show no difference between the control conditions using the amino acids and the substituted conditions. The three critical quality attributes are glycosylation patterns, antibody aggregation and fragmentation as well as charge variants.

It has further been found that the keto acids and/or derivatives thereof, especially the keto acids and/or salts of leucine, isoleucine and valine, preferably 4-Methyl-2-oxopentanoic acid, 3-methyl-2-oxopentanoic acid and/or salts thereof are suitable for stabilizing liquid cell culture media formulations. Liquid media comprising the said components show a lower change in color when stored over three months at either room temperature or at 4° C. with or without light exposure compared to media comprising no 4-Methyl-2-oxopentanoic acid, 3-methyl-2-oxopentanoic acid and/or salts thereof but the same amount of the corresponding amino acid. They also showed a reduced precipitation.

This effect can be reached when substituting the corresponding amino acids, e.g. isoleucine and/or leucine with the corresponding keto acid and/or derivatives thereof. It can also be reached when adding the corresponding keto acid, e.g. 4-Methyl-2-oxopentanoic acid, 3-methyl-2-oxopentanoic acid and/or derivatives thereof to a cell culture media formulation comprising leucine and/or isoleucine. That means the keto acids and/or derivatives thereof can be used as medium stabilizers irrespective of the media composition. Suitable concentrations in the liquid formulation are at least 20 mM, preferably between 30 and 600 mM.

The powdered cell culture media of the present invention are preferably produced by mixing all components and milling them. The mixing of the components is known to a person skilled in the art of producing dry powdered cell culture media by milling. Preferably, all components are thoroughly mixed so that all parts of the mixture have nearly the same composition. The higher the uniformity of the composition, the better the quality of the resulting medium with respect to homogenous cell growth.

The milling can be performed with any type of mill suitable for producing powdered cell culture media. Typical examples are ball mills, pin mills, fitz mills or jet mills. Preferred is a pin mill, a fitz mill or a jet mill, very preferred is a pin mill.

A person skilled in the art knows how to run such mills.

A large scale equipment mill with a disc diameter of about 40 cm is e.g. typically run at 1-6500 revolutions per minute in case of a pin mill, preferred are 1-3000 revolutions per minute.

The milling can be done under standard milling conditions resulting in powders with particle sizes between 10 and 300 μm, most preferably between 25 and 120 μm.

The size of a particle means the diameter of the particle. The particle diameter is determined by laser light scattering. Using this technique, the particle size is reported as a volume equivalent sphere diameter.

A particle size range gives the range of the particle size which 75% or more, preferably 90% or more of the particles have. That means if the particle size is between 25 and 120 μm at least 75% of the particles have a particle size between 25 and 120 μm.

Preferably, all components of the mixture which is subjected to milling are dry. This means, if they comprise water, they do only comprise water of crystallization but not more than 10%, preferably not more than 5% most preferred not more than 2% by weight of unbound or uncoordinated water molecules.

In a preferred embodiment, the milling is performed in an inert atmosphere. Preferred inert protective gas is nitrogen.

In another preferred embodiment, all components of the mixture are freezed prior to milling. The freezing of the ingredients prior to the milling can be done by any means that ensures a cooling of the ingredients to a temperature below 0° C. and most preferably below −20° C. In a preferred embodiment the freezing is done with liquid nitrogen. This means the ingredients are treated with liquid nitrogen, for example by pouring liquid nitrogen into the container in which the ingredients are stored prior to introduction into the mill. In a preferred embodiment, the container is a feeder. If the container is a feeder the liquid nitrogen is preferably introduced at the side or close to the side of the feeder at which the ingredients are introduced.

Typically the ingredients are treated with the liquid nitrogen over 2 to 20 seconds.

Preferably the cooling of the ingredients is done in a way that all ingredients that enter into the mill are at a temperature below 0° C., most preferred below −20° C.

In a preferred embodiment, all ingredients are put in a container from which the mixture is transferred in a feeder, most preferred in a metering screw feeder. In the feeder the ingredients are sometimes further mixed—depending on the type of feeder—and additionally cooled. The freezed mixture is then transferred from the feeder to the mill so that the mixture which is milled in the mill preferably still has a temperature below 0° C., more preferred below −20° C.

Typically the blending time, that means the residence time of the mixture of ingredients in the feeder is more than one minute, preferably between 15 and 60 minutes.

A metering screw feeder, also called dosage snail, is typically run at a speed of 10 to 200 revolutions per minute, preferably it is run at 40 to 60 revolutions per minute.

Typically, the temperature of the mill is kept between −50 and +30° C. In a preferred embodiment, the temperature is kept around 10° C.

The oxygen level during milling preferably is below 10% (v/v).

The process can be run e.g. batch-wise or continuously. In a preferred embodiment the process according to the present invention is done continuously by, over a certain time, permanently filling the mixture of ingredients into a feeder for cooling and permanently filling cooled mixture from the feeder into the mill.

The present invention is further directed to a process for culturing cells by
a) providing a bioreactor
b) providing a liquid cell culture medium comprising at least one alpha keto acid out of the group of 4-Methyl-2-oxopentanoic acid, 3-methyl-2-oxopentanoic acid, alpha-ketoisovaleric acid, phenylpyruvic acid and alpha keto gamma methylthiobutyric acid and/or derivatives thereof, preferably in a concentration of above 10 mM.
c) mixing the cells to be cultured with the liquid cell culture medium
d) incubating the mixture of step b)

In a preferred embodiment the cells are CHO cells.

In one embodiment the liquid cell culture medium provided in step b) is a liquid cell culture medium in which one or more of the amino acids isoleucine, leucine, valine, phenylalanine and methionine are partially or preferably fully substituted by the corresponding keto acid selected from the group of 4-Methyl-2-oxopentanoic acid, 3-methyl-2-oxopentanoic acid, alpha-ketoisovaleric acid, phenylpyruvic acid and alpha keto gamma methylthiobutyric acid and/or derivatives thereof.

In a preferred embodiment the liquid cell culture medium of step b) is provided by dissolving a dry powder or dry, granulated medium according to the present invention in a solvent as described above.

A bioreactor is any vessel or tank in which cells can be cultured. Incubation is typically done under suitable conditions like suitable temperature etc. A person skilled in the art is aware of suitable incubation conditions for supporting or maintaining the growth/culturing of cells.

It has been found that the present invention is also very suitable for the preparation of feed media. Due to the limitations in the availability of certain amino acids especially in the concentrations necessary for feed media the concentration of feed media is limited due to solubility problems.

Consequently there is a need for feed media that comprise all needed components in one feed and at high concentrations. In addition the pH of the feed should not negatively influence the cell culture, i.e. the pH of the liquid feed should be below 8.5, preferably between 6.5 and 7.8.

It has been found that by partially or preferably fully substituting the amino acids isoleucine, leucine, valine, phenylalanine and methionine by the corresponding keto acids and/or derivatives, preferably the salts thereof the solubility of the resulting dry powder medium is improved. This offers the possibility to produce liquid media with a higher concentration of ingredients so that the same amount of ingredients can be added to the cell culture in a smaller amount of liquid but nevertheless at a suitable pH which is preferably below 8.5. The higher concentrated feed media comprising the keto acids can be used without any negative effect and sometimes even positive effect on the cell growth and/or productivity as well as on the stability of the liquid medium.

The present invention is thus also directed to a feed medium either in form of a powdered medium or after dissolution in form of a liquid medium.

The resulting liquid medium comprises at least one keto acid selected from the group of 4-Methyl-2-oxopentanoic acid, 3-methyl-2-oxopentanoic acid, alpha-ketoisovaleric acid, phenylpyruvic acid and alpha keto gamma methylthiobutyric acid and/or derivatives thereof in a concentration of more than 10 mM, preferably between 20 and 600 mM and preferably has a pH of 8.5 or less.

In a preferred embodiment, the pH is between 6.7 and 8.4.

The present invention is also directed to a fed batch process for culturing cells in a bioreactor by
Filling into a bioreactor cells and an aqueous cell culture medium
Incubating the cells in the bioreactor
Continuously over whole time of the incubation of the cells in the bioreactor or once or several times within said incubation time adding a cell culture medium, which is in this case a feed medium, to the bioreactor

whereby the feed medium preferably has a pH of less than pH 8.5 and comprises at least one keto acid selected from the group of 4-Methyl-2-oxopentanoic acid, 3-methyl-2-oxopentanoic acid, alpha-ketoisovaleric acid, phenylpyruvic acid and alpha keto gamma methylthiobutyric acid and/or derivatives thereof.

Preferably, the feed medium comprises the one or more keto acids and/or derivatives thereof in a concentration of more than 10 mM, preferably between 20 and 600 mM. Preferably the feed medium comprises 4-Methyl-2-oxopentanoic acid, 3-methyl-2-oxopentanoic acid and/or alpha-ketoisovaleric acid and/or salts thereof, most preferred the sodium salts. Typically a feed medium comprises between 50 and 400 g/l of solid ingredients that are dissolved in the solvent.

In a preferred embodiment, in the process of the present invention the feed medium that is added during the incubation either continuously or once or several times within said time to the bioreactor always has the same composition. In a preferred embodiment the cells are CHO cells.

The present invention is further illustrated by the following figures and examples, however, without being restricted thereto.

The entire disclosure of all applications, patents, and publications cited above and below are hereby incorporated by reference.

### EXAMPLES

The following examples represent practical applications of the invention.

### Example 1: Keto Acids have an Increased Solubility Compared to their Respective Amino Acids in Water

The maximum solubility of five exemplary amino acids was compared with the solubility of their respective keto acids or salts thereof in water at 25° C. through the preparation of a saturated solution. After sedimentation, the solution was dried using infrared (120° C., 120 min) and the residual mass was determined in g/kg.

As shown in FIG. 1, the solubility of keto acids and salt thereof is significantly higher when compared to the solubility of the respective amino acid in water. To exclude that the increase in solubility is due to the sodium salt form of the keto acid, separate experiments were performed to compare the solubility of Leu, Leu sodium salt and keto Leu sodium salt. The maximum solubilities obtained in water were 22.1, 86.0 and 313.7 g/kg respectively indicating that, as expected, the formation of a sodium salt increases already the solubility of Leu but the increase in solubility obtained with the keto acid is significantly more important and thus can't be due to only the salt form.

### Example 2: Maximum Solubility of Keto Acids when Compared to their Respective Amino Acids in 4Feed Depleted in Ile and Leu

Increasing amounts of the keto acids and salts thereof were added to a cell culture feed formulation (Cellvento® 4Feed, MilliporeSigma) depleted in Ile and Leu. Similarly, increasing amount of Ile and Leu were added to the same feed formulation as a control. The total concentration of this feed formulation was 125 g/L and the pH was 7.0+/−0.2. In small scale experiments, after each addition of either the amino acid or the keto acid, the feed was agitated for 10 mins and turbidity was measured. The experiments were performed at room temperature (25° C.). The maximum solubility of Ile in Cellvento® 4Feed depleted in Ile/Leu was found to be approximatively 105 mM whereas for keto Ile, the maximum tested concentration of 635 mM was still soluble with a turbidity value below 5 NTU (see FIG. 1). This indicates that keto Ile is at least 6 times more soluble than Ile in 4Feed depleted in Ile/Leu.

The maximum solubility of Leu in Cellvento® 4Feed depleted in Ile and Leu was found to be approximatively 90 mM whereas for keto Leu, the maximum soluble concentration (with a turbidity value below 5 NTU) was 240 mM (see FIG. 2). This indicates that keto Leu is 2.6 times more soluble than Leu in 4Feed depleted in Ile/Leu.

### Example 3: The Use of Keto Acids Enables the Concentration of Cell Culture Media Formulations at Neutral pH

The maximum solubility of Cellvento® 4Feed was determined by dissolving increasing amounts of feed dry powder media in water until precipitation was detected visually. For each condition, the feed was stirred for about 30 min, the pH was adjusted to 7.0+/−0.2. and the solution was stirred for another 10 min for equilibration. Osmolality and turbidity were measured (see FIG. 3). The data indicate that already a 1.2× concentrate of this formulation is not soluble since particles can be detected in suspension and the turbidity is largely above the limit of 5 NTU.

Since Ile and Leu have been identified as the first limiting amino acids for the concentration of the Cellvento® 4Feed formulation, a new backbone feed depleted in Ile and Leu was produced (4Feed—Ile/Leu).

The maximum concentration of this feed with or without supplementation with keto Leu and keto Ile was determined by dissolving increasing amounts of feed dry powder media in water until precipitation was detected visually. For each condition, the feed was stirred for about 30 min, the pH was adjusted to 7.0+/−0.2. and the solution was stirred for another 10 min for equilibration. Turbidity was measured and a limit of 5 NTU was considered soluble.

Results indicate that the maximum solubility of the Ile/Leu depleted Cellvento® 4Feed was approximatively 228 g/L. Upon addition of keto Leu and keto Ile, the maximum solubility was obtained between 216 g/L and 228 g/L of the depleted dry powder media supplemented with a combined amount of keto Leu and keto Ile of 36 g/L to 38 g/L (molar equivalent to the theoretical amount of Ile and Leu in that concentrate) yielding a total concentration of 252 g/L-266 g/L for the formulation containing both keto Leu and keto Ile. Considering that Cellvento® 4Feed has a concentration of 130 g/L, this represent an increase in concentration of 100% when Ile and Leu are replaced by keto Ile and keto Leu.

The data indicate that it is possible to concentrate that formulation until at least 2× (265 g/L) since no particles can be detected in suspension and the turbidity is below 5 NTU (see FIG. 4).

### Example 4: The Keto Acids of Leu and Ile can Stabilize Cell Culture Media Formulations

The stability of a feed containing Ile and Leu (Cellvento® 4Feed) was compared to the stability of the same feed depleted in Ile/Leu and with supplementation of either keto Leu or keto Ile. The feeds were prepared according to the standard protocol. The final pH was 7.0+/−0.2 and the feeds were stored at 4° C. or RT protected or exposed to light. The change in color of the formulations were monitored during 90 days by measuring the absorbance in the range from 300 nm to 600 nm (intervals of 5 nm). Conditions were compared by calculating the area under the curve (AUC) over time (between D0 and D90) of the baseline corrected area under the curve of the absorbance scan (300 nm-600 nm).

As shown in FIG. 5A, the feed containing Ile and Leu in the control condition became darker (AUC increased from 350 to 7000) with increasing temperature or light exposure. At 4° C., the AUC was decreased by 27% and 8% in the light protected and light exposed conditions respectively when Leu was replaced by keto Leu. At RT, the decrease was even more pronounced with a reduction of 31% (light protected) and 37% (light exposed) of the AUC respectively in the keto Leu condition. This indicates that the replacement of Leu by keto Leu can significantly decrease the change in color observed in feeds over time.

Results obtained for keto Ile are presented in FIG. 5B. As for keto Leu, reduction in the AUC was observed when Ile was replaced with keto Ile. At 4° C., the AUC was decreased by 33 and 68% in the light protected and light exposed conditions respectively. At RT, no decrease was seen in the light protected condition but a decrease of 38% was observed in the light exposed condition indicating that the replacement of Ile by keto Ile can significantly decrease the change in color observed in feeds over time.

Overall our results indicate that the replacement of amino acids by their keto acid or salt thereof can lead to a stabilization resulting in a lower change in color when stored for 3 months at either 4° C. or RT with or without light exposure.

In addition, when using keto Leu instead of Leu in the feed, the precipitation of the feed was delayed. To observe precipitation, the 50 mL falcon tubes were turned back to see possible sedimentation on the bottom of the tube and pictures were taken. At 4° C., none of the conditions precipitated but at RT light protected, the control condition precipitated between D49 and D70 whereas no precipitation was observed in the condition containing keto Leu. A complete inhibition of precipitation was not observed at RT exposed to light but the precipitation was delayed in the keto Leu condition. Whereas precipitation was observed starting at D49 for the control condition, initial precipitation appeared at D70 for the keto Leu condition. During the following days, the amount of precipitate as well as the color intensity of the precipitate was lower in the keto Leu containing condition indicating that the stability was slightly enhanced for the keto Leu formulation at RT light exposed too.

Finally, the amount of ammonium ions formed during storage of the feed containing keto acids at 4° C. or RT was lower when compared to the feed containing the normal amino acids. To be able to evaluate the formation of the NH₃ over the entire period of the stability study, the AUC of the NH₃ concentration was calculated for the timeframe of 3 months to compare the conditions.

Results for keto Leu are presented in FIG. 6A and indicate a lower ammonia formation when compared to the control condition. 10 and 19% less ammonia was produced in the keto Leu condition compared to the control when feeds were stored at 4° C. light protected and light exposed respectively. At RT, the same trend was observed with a reduced ammonia level of 15 and 5% respectively when the feed was stored light protected and light exposed for 3 months.

Similar results were obtained for keto Ile (FIG. 6B) and indicate a lower ammonia formation when compared to the control condition. 21 and 24% less ammonia was produced in the keto Ile condition compared to the control when feeds were stored at 4° C. light protected and light exposed respectively. At RT, the same trend was observed with a reduced ammonia level of 28 and 25% respectively when the feed was stored light protected and light exposed for 3 months.

### Example 5: Keto Ile and Keto Leu can Replace their Respective Amino Acids in the Feed and Increase Specific Productivity. Cell Culture Results with a CHOK1GS Clone Producing an IgG1

For cell culture experiments, a CHOK1GS suspension cell line expressing a human IgG1, was used. Cells were cultivated in quadruplicate in Cellvento 4CHO medium (Merck Darmstadt, Germany) using 50 mL spin tubes with a starting culture volume of 30 mL and a seeding density of 2×10⁵ cells/mL. Incubation was carried out at 37° C., 5% CO₂, 80% humidity and an agitation of 320 rpm. The keto acids were added in the Feed (4Feed depleted in Ile and leu) instead of their respective amino acids. The pH of all the feeds was neutral (pH 7.0+/−0.2). The positive control contained the normal amino acids whereas the negative control contained the feed depleted in the respective amino acid and without addition of keto acid. Feeding was carried out at days 3, 5, 7, 10 and 14 at the following v/v ratios (3, 3, 6, 3 and 3%). Glucose was quantified daily and adjusted to 6 g/L using a 400 g/L glucose solution. The experiment was repeated at least 3 times.

The viable cell density (VCD) and viability were evaluated with a Vi-CELL XR (Beckman Coulter, Fullerton, Calif.). Metabolite concentrations were monitored using a Cedex Bio HT (Roche Diagnostics, Mannheim, Germany) based on spectrophotometric and turbidometric methods. Quantification of amino acids was carried out via UPLC after derivatization with the AccQ⋅TagUltra® reagent kit. Derivatization, chromatography and data analysis were carried out following the supplier recommendations (Waters, Milford, Mass.).

The productivity per cell per day was calculated daily by dividing the titer by the corrected integral VCD to take into account the dilution resulting from feeding. The overall specific productivity was determined by calculating the slope from the linear regression between titer and corrected integral VCD.

When looking at the viable cell density (FIG. 7A), both keto derivatives lead to slightly lower maximum VCD compared to the control but the titer obtained after day 11 (FIG. 7B) was slightly higher than the control condition, indicating an overall higher specific productivity (FIG. 8). The negative control for which the feed was depleted in Leu and Ile showed a rapid decrease in VCD after day 7 and most importantly a very limited IgG titer, indicating that Leu and Ile are critical to support IgG production by CHO cells.

NH₃ is an undesired metabolite that is produced over the course of the fed-batch process. The amount of NH₃ produced during the 17-days fed-batch process in the keto Leu and keto Ile conditions (FIG. 9A) was significantly reduced compared to the control containing Leu and Ile, indicating that either a significant part of the ammonia is generated from the oxidative deamination of Leu and Ile, or that the presence of keto acids in the bioreactor media is promoting the usage of free NH₃ as a building block to generate amino acids through amination.

The concentration of the amino acids was determined in spent media. In the condition in which Leu has been replaced with keto Leu, the concentration of Leu in the spent medium (FIG. 9B) was slightly lower than in the positive control (containing Leu and Ile) but the evolution with time shows concentration increases between the feeding day and the subsequent day, indicating that Leu can be produced very quickly from keto Leu. In the condition where Ile has been replaced with keto Ile (FIG. 10A), the concentration of Ile that was detected over time was significantly lower than the Ile concentration in the positive control indicating that either the conversion of keto Ile to Ile is slow or that another product is formed from keto Ile in culture. The comparison of the keto Ile condition with the negative control in which the feed has been depleted in Ile and Leu indicate nevertheless that Ile can be produced from keto Ile in that fed-batch. In addition, careful analysis of the chromatograms allowed the identification of a new peak corresponding to allo-Ile (FIG. 10B), which increased with time.

The quality of the antibody produced in the control fed-batch process (with feed containing Ile and Leu) was compared to the quality of the antibody produced with feed depleted in either Leu and Ile and supplemented with either keto Leu or keto Ile.

The antibody was purified from the cell culture supernatant using protein A PhyTips® (PhyNexus Inc, San Jose, Calif.). Glycosylation patterns were analyzed by capillary gel electrophoresis with laser-induced fluorescence (CGE-LIF) after derivatization using the GlykoPrep®-plus Rapid N-Glycan Sample Preparation kit with 8-aminopyrene-1,3,6-trisulfonic acid trisodium (APTS) (Prozyme, Hayward, Calif.) according to the manufacturer's instructions. Briefly, the purified antibody was denatured and immobilized, and the glycans were released from the antibody by digestion with N-Glycanase® followed by labeling with APTS for 60 min at 50° C. After a cleaning step to remove the remaining APTS, the relative amounts of glycans were determined using the Pharmaceutical Analysis System CESI8000 Plus (Sciex, Washington, USA) with a LIF detector (Ex: 488 nm, Em: 520 nm). Separation was performed in a polyvinyl alcohol-coated capillary (total length: 50.2 cm, inner diameter: 50 μm) and filled with the carbohydrate separation buffer from the carbohydrate labeling kit (Beckman Coulter, Brea, USA). The capillary surface was first rinsed with separation buffer at 30 psi for 3 min. Inlet and outlet buffer vials were changed every 20 cycles. Samples were introduced by pressure injection at 0.5 psi for 12 s followed by a dipping step for 0.2 min to clean the capillary tips. Separation was finally performed at 20 kV for 20 min with a 0.17 min ramp applying reverse polarity. Peaks were identified according to their individual migration times and integrated according to the following parameters: peak width 0.05, threshold 10,000 and shoulder sensitivity 9,999.

Antibody aggregation and fragmentation were measured using size exclusion chromatography on an Water Acquity UPLC system using a TSKgel SuperSW3000 column (Tosoh Bioscience). The mobile phase was 0.05 M Sodium phosphate, 0.4 M Sodium perchlorate, pH 6.3 and the flow rate was 0.35 mL/min. The sample concentration was adjusted to 1.0 mg/mL after the IgG purification using the storage buffer and the detection was performed using the absorbance at 214 nm.

Charge variants were measured on a Capillary Electrophoresis CESI 8000 (Beckman Coulter/Sciex) using cIEF according to the manufacturer's instructions. The sample concentration was adjusted to a concentration of 1.5 mg/mL after the IgG purification using the storage buffer. Prior to the measurement, the samples were mixed with a master mix which contained different pH markers, a cathodic/anodic stabilizer, 3M Urea cIEF gel and Pharmalyte.

Results obtained for glycosylation (FIG. 11), high and low molecular weight species (FIG. 12A) and charge variants (FIG. 12B), indicate no difference between the control condition and the conditions where Ile and Leu have been exchanged with keto Ile and keto Leu, indicating that the amino acid exchange has no impact on the 3 critical quality attributes of the IgG1 produced in this study.

### Example 6: Confirmation of Keto Leu Performance with CHODG44 and CHOK1 Clones Producing an IgG1

The applicability of the technology of the present invention for different bioprocesses was demonstrated by performing fed-batch experiments with other types of CHO cells: CHODG44 and CHOK1 (non GS) for keto Leu as an example. Results for a DG44 cell line (FIG. 13) indicate a lower VCD and a slightly lower IgG titer for the keto Leu condition compared to the control. Nevertheless, the overall specific productivity was slightly increased for the process using keto Leu. The spend media data show that for this cell line too, the Leu concentration in the keto Leu condition was nearly similar to the Leu concentration in the control, confirming that Leu can be produced very quickly from keto Leu in that cell line as well.

FIG. 13: Performance of the keto Leu containing process compared to the control for a CHODG44 cell line expressing an IgG1.

FIG. 14: Performance of keto Leu containing process compared to the control for a CHOK1 non GS cell line expressing an IgG1.

### Example 7: Performance in Batch with Different Seeding Densities and Different Leu/Keto Leu Ratios

Our fed-batch spent media results obtained with 3 different CHO cell lines with keto Ile and keto Leu indicate that the formation of Ile, allo-Ile and Leu from the keto acids is fairly quick. This indicates that keto acids may also be used to increase the solubility of batch and perfusion media since they are likely to be readily available from the start of the culture.

To confirm that this is applicable in CHO systems, keto Leu or keto Ile were used as replacement for Leu and Ile respectively in a cell culture medium (Cellvento® 4CHO). A Leu/Ile depleted version of the formulation was produced and equimolar concentrations of keto Leu vs Leu and keto Ile vs Ile were used (FIG. 15). Cell growth and viability of a CHOK1GS cell line were monitored over several weeks in serial passaging experiments to make sure that the growth is not due to residual amounts of Leu or Ile. A batch experiment with a seeding density of 0.2 million cells/mL was designed and IgG production was measured over time. The production of the amino acids was followed with time using amino acids quantification in the spent medium.

In a similar way, batch experiments with higher cell seeding densities were performed in media containing different ratios of Leu/keto Leu to understand which ratios are preferable when starting with higher cell densities (FIG. 16). The analytic used was the same as described above.

Results of the serial passaging indicate that CHOK1GS cells cannot grow in a medium depleted in Ile and Leu since no growth was observed during the first days of culture and the viability decreased very drastically. In contrast, a continuous growth was observed when Leu or Ile were replaced by their corresponding keto acids. Overall, the maximum viable cell density observed for each passage was slightly lower than in the control condition containing Ile and Leu, indicating that a small amount of Leu and Ile might be needed to obtain comparable performance than in the control condition. This amount can be determined experimentally very easily by testing media containing different ratios of Ile/Leu and keto Ile/keto Leu.

In batch experiments, the performance was comparable between the control condition and the keto Leu condition, indicating that CHOK1GS cells can grow when Leu is replaced by the molar equivalent of keto Leu (FIG. 15A). In that condition, a similar amount of IgG was detected at day 7 and day 10 (FIG. 15B). In contrast, the growth and IgG concentration after day 5 was slightly impaired when Ile was replaced with keto Ile, indicating that a small amount of Ile might be necessary to obtain a similar growth and titer over time than with the control in batch conditions. This amount can be determined experimentally very easily by testing media containing different ratios of Ile and keto Ile. Alternatively, a higher molar keto Ile concentration compared to the Ile concentration might be tested.

The difference between the performance of keto Ile and keto Leu might be explained by looking at the formation of Ile, allo-Ile and Leu in the spent medium. Whereas 34% of the initial keto Leu concentration was detected in form of Leu on day 3, only 21% of the initial keto Ile concentration was detected as Ile on day 3. In addition, 35% of the initial keto Ile concentration was detected in the form of allo-Ile up to day 10. This indicates that the amination of keto Leu to Leu by cells is more efficient than the amination of keto Ile to Ile due to the concomitant formation of allo-Ile which might not be used to the same extend than Ile by cells.

Finally, batch experiments with higher cell densities were performed to determine whether keto Leu is readily available when starting with high seeding densities or if a minimum concentration of free leucine has to be present to support growth and productivity in these conditions. For this experiment, a CHOK1GS cell line was seeded at 0.3, 0.6 or 1.10\hat{ }6 cells/mL in a medium containing 0, 25, 50, 75, or 100% of keto Leu, the rest being added in form of Leucine.

Results indicate an increased in growth and titer with increased seeding density as expected. Between the different ratios keto Leu/Leu, the maximum VCD was observed with 100% keto leu exchange with the highest seeding density of 1.10\hat{ }6 cells/mL. When the cells where seeded at 0.6.10\hat{ }6 cells/ml and 0.3.10\hat{ }6 cells/mL, the highest VCD was observed for a ratio keto Leu:leu of 1:1 (50% of keto Leu and 50% of Leu). Regarding titer, no significant difference was seen for a seeding at 0.3 and 1.10\hat{ }6 cells/mL while a slight trend was seen with an increase in IgG concentration with higher ratios of leu/keto Leu for the seeding at 0.6.10\hat{ }6 cells/mL. This difference might not be significant.

### Example 8. Performance of Other Keto Acids Vs their Respective Amino Acid in FB Culture

Other keto acids have been tested as replacement for their respective amino acids in FB experiments. A very similar behavior compared to Ile and Leu was observed when Val was replaced by keto Val in the feed (FIG. 17). Indeed, similar VCD and titer were observed compared to the positive control whereas the feed depleted in Val leads to a drastic decrease in the VCD, as well as a very low titer after day 7. The NH₃ concentration was also lower when the keto acid was used indicating that for Val, too, the usage of the respective keto acids can lead to less NH₃ during fed-batch culture. Overall, this indicates that keto Val as a member of the branched chain keto acids, is most likely exhibiting the same behavior as keto Leu and keto Ile and is likely to be aminated very quickly in cell culture. Due to the structural similarity to keto Ile and keto Leu, the effect of keto Val on overall feed concentration and feed stability is similar to the other branched chain keto acids. 6× higher solubility when compared to Val was confirmed in water.

For phenylalanine (Phe) and its respective keto acid phenylpyruvate (FIG. 18), the amination reaction to build Phe in cell culture seemed to be slower than the amination reactions occurring for branched chain keto acids. Indeed, when Phe was replaced with the equivalent molar concentration of phenylpyruvate, spent media data revealed that although more Phe was found in the supernatant compared to the negative control (Feed depleted in Phe), the amount formed was not sufficient to support the same growth and titer than in the control condition. A significant lower VCD was observed after day 5 and a final reduction of titer of 20% was observed. Following that result, a condition where 2× the molar equivalent of Phe was used as a concentration of phenylpyruvate in the feed. Results indicate that the increase in the amount of phenylpyruvate could restore the VCD, titer as well as a very similar amount of Phe in the spent medium. These data confirm that also Phe can be replaced by its keto acid, but concentration adjustments may be needed to cope with the slower pace of the amination reaction.

1. A dry powder or dry, granulated cell culture medium comprising at least one alpha keto acid out of the group of 4-Methyl-2-oxopentanoic acid, 3-methyl-2-oxopentanoic acid, alpha-ketoisovaleric acid, phenylpyruvic acid and alpha keto gamma methylthiobutyric acid and/or derivatives thereof.

2. A dry powder or dry, granulated cell culture medium according to claim 1, whereby the one or more alpha keto acids out of the group of 4-Methyl-2-oxopentanoic acid, 3-methyl-2-oxopentanoic acid, alpha-ketoisovaleric acid, phenylpyruvic acid and alpha keto gamma methylthiobutyric acid and/or derivatives thereof are present in an amount so that the concentration in the liquid medium obtained after dissolution of the dry powder or dry, granulated cell culture medium for each of the alpha keto acids is above 10 mM.

3. The dry powder or dry, granulated cell culture medium according to claim 1, whereby the dry powder or dry, granulated cell culture medium does not comprise the corresponding amino acid.

4. The dry powder or dry, granulated cell culture medium according to claim 1, whereby the medium comprises the sodium salt of one or more of the alpha keto acids out of the group of 4-Methyl-2-oxopentanoic acid, 3-methyl-2-oxopentanoic acid, alpha-ketoisovaleric acid, phenylpyruvic acid and alpha keto gamma methylthiobutyric acid.

5. The dry powder or dry, granulated cell culture medium according to claim 1, whereby, the dry powder or dry, granulated cell culture medium comprises one or more of the sodium salt of the alpha keto acids selected from 4-Methyl-2-oxopentanoic acid, 3-methyl-2-oxopentanoic acid and/or alpha-ketoisovaleric acid.

6. A method for producing a dry powder cell culture medium according to claim 1 by
a) mixing at least one alpha keto acid out of the group of 4-Methyl-2-oxopentanoic acid, 3-methyl-2-oxopentanoic acid, alpha-ketoisovaleric acid, phenylpyruvic acid and alpha keto gamma methylthiobutyric acid and/or derivatives thereof with the other components of the cell culture medium
b) subjecting the mixture of step a) to milling

7. A process for culturing cells by
a) providing a bioreactor
b) mixing the cells to be cultured with a liquid cell culture medium prepared by dissolving a dry powder or dry, granulated medium according to claim 1 in a solvent
c) incubating the mixture of step b).

8. A fed batch process for culturing cells in a bioreactor by
Filling into a bioreactor cells and an aqueous cell culture medium
Incubating the cells in the bioreactor
Continuously over whole time of the incubation of the cells in the bioreactor or once or several times within said incubation time adding a cell culture medium, which is in this case a feed medium, to the bioreactor whereby the feed medium is prepared by dissolving a dry powder or dry, granulated medium according to claim 1 in a solvent.

9. The fed batch process according to claim 8, whereby the feed medium comprises at least 4-Methyl-2-oxopentanoic acid, 3-methyl-2-oxopentanoic acid, alpha-ketoisovaleric acid and/or salts thereof in a concentration between 12 and 600 mmol/l

10. A method for stabilizing a liquid cell culture medium comprising including in the medium at least 20 mM 4-Methyl-2-oxopentanoic acid, 3-methyl-2-oxopentanoic acid, alpha-ketoisovaleric acid, phenylpyruvic acid and/or alpha keto gamma methylthiobutyric acid and/or derivatives thereof and whereby the resulting medium shows less color change and/or less precipitation upon storage over 90 days at 4° C. or at room temperature compared to a medium of the otherwise same composition which lacks the 4-Methyl-2-oxopentanoic acid and/or 3-methyl-2-oxopentanoic acid and/or derivatives thereof or in which the 4-Methyl-2-oxopentanoic acid and/or 3-methyl-2-oxopentanoic acid and/or derivatives thereof have been substituted by corresponding amino acids and/or derivatives thereof.

11. A method for improving the solubility of a dry powder or dry, granulated cell culture medium by fully or partially substituting one or more of the amino acids isoleucine, leucine, valine, phenylalanine and methionine by the corresponding keto acid selected from the group of 4-Methyl-2-oxopentanoic acid, 3-methyl-2-oxopentanoic acid, alpha-ketoisovaleric acid, phenylpyruvic acid and alpha keto gamma methylthiobutyric acid and/or derivatives thereof.

12. The method according to claim 11, whereby at least 50% (molar ratio) of the respective amino acid is substituted by the corresponding alpha keto acid and/or derivatives thereof.

13. The method according to claim 11, whereby the method involves providing the dry powder or dry, granulated cell culture medium in which compared to the original composition at least 50% (molar ratio) of the respective amino acid is substituted by the corresponding alpha keto acid and/or derivatives thereof and dissolving said medium in a solvent whereby dissolution occurs faster and/or in less of the solvent compared to a medium of the original composition which is the otherwise identical composition in which the amino acids have not been substituted.
